SEQUENCE LISTING (14 sequences)

SEQ ID NO: 1 - protein, 18 aa, Artificial
  Synthetic
TRDHFSGPDNDFWSGSDF

SEQ ID NO: 2 - protein, 20 aa, Artificial
  Synthetic
VRHRPRGAFPLPYTHNGMDL

SEQ ID NO: 3 - protein, 16 aa, Artificial
  Synthetic
ARYHRHFIRGPLSFDY

SEQ ID NO: 4 - protein, 12 aa, Artificial
  Synthetic
ARGTRGGSTLDS

SEQ ID NO: 5 - protein, 11 aa, Artificial
  Synthetic
ARLKLRGAFDF

SEQ ID NO: 6 - protein, 13 aa, Artificial
  Synthetic
VTTRRGSHYKDDY

SEQ ID NO: 7 - protein, 11 aa, Artificial
  Synthetic
ARGFWSGLVDS

SEQ ID NO: 8 - protein, 19 aa, Artificial
  Synthetic
ARETKRGLSLPYTHNGMDV

SEQ ID NO: 9 - protein, 8 aa, Artificial
  Synthetic
RRYGSSRT

SEQ ID NO: 10 - protein, 9 aa, Artificial
  Synthetic
QQLKRFPLT

SEQ ID NO: 11 - protein, 10 aa, Artificial
  Synthetic
QSQAFSPRFL

SEQ ID NO: 12 - protein, 10 aa, Artificial
  Synthetic
QNQGFSPRFF

SEQ ID NO: 13 - protein, 9 aa, Artificial
  Synthetic
QQLSTYPRT

SEQ ID NO: 14 - protein, 8 aa, Artificial
  Synthetic
QQYGGSPW

(19) United States
(12) Patent Application Publication
(10) Pub. No.: US 2007/0292390 A1
(43) Pub. Date: Dec. 20, 2007

(54) Broadly Cross-Reactive Hiv-1 Neutralizing Human Monoclonal Antibodies

(76) Inventors: Dimiter S. Dimitrov, Frederick, MD (US); Mei-Yun Zhang, Frederick, MD (US)

Correspondence Address: LEYDIG, VOIT & MAYER, LTD., TWO PRUDENTIAL PLAZA, SUITE 4900, 180 NORTH STETSON AVENUE, CHICAGO, IL, 60601-6731, US

(21) Appl. No.: 11/718,202

(22) PCT Filed: Oct. 28, 2005
(86) PCT No.: PCT/US05/39175

§ 371 Date: Aug. 10, 2007

Related U.S. Application Data
(60) Provisional application No. 60/623,394, filed on Oct. 29, 2004.

(51) Int. Cl. A61K 39/42; A61K 31/7105; A61K 38/20; C07K 16/08; C12N 15/12; G01N 33/53; C12N 15/63; C12N 1/00; A61P 31/18; A61K 38/21

(52) U.S. Cl. 424/85.2; 424/160.1; 424/85.4; 435/243; 435/320.1; 435/5; 436/86; 530/388.35; 530/391.1; 536/23.5

(57) ABSTRACT

The invention provides polypeptides that bind with an epitope of the gp41 subunit of the HIV-1 envelope glycoprotein, as well as polypeptides comprising the aforementioned epitopes. The invention also provides methods of inhibiting an HIV infection in a mammal using the polypeptides of the invention, as well as compositions comprising the polypeptides, nucleic acid molecules encoding the polypeptides, and host cells and vectors comprising the nucleic acid molecules. A method of isolating antibodies that bind with an epitope of the gp41 subunit of the HIV-1 envelope glycoprotein using competitive antigen panning (CAP) is also provided. The invention also features the use of the polypeptides to detect the presence of HIV in a mammal, and epitopes that can be used as vaccine immunogens.

### FIELD OF THE INVENTION

This invention pertains to broadly neutralizing antibodies against Human Immunodeficiency Virus, and methods of using the same.

### BACKGROUND OF THE INVENTION

The Human Immunodeficiency Virus (HIV) is the causative agent of Acquired Immunodeficiency Syndrome (AIDS). HIV rapidly undergoes genetic changes to escape from the patient's immune system response. Identification of potent broadly cross-reactive human monoclonal antibodies to HIV has major implications for development of HIV inhibitors, vaccines, and tools for understanding mechanisms of HIV entry.

The binding of the HIV-1 envelope glycoprotein to CD4 and coreceptors initiates a series of conformational changes that lead to viral entry into cells (see, e.g., Moulard et al., PNAS, 99(10): 6913-6918 (2002)). HIV-1 envelope glycoprotein (Env) is composed of two subunits, gp120 and gp41. gp120 is highly variable and immunogenic. gp41 is conserved, but unstable in the absence of the other subunit, gp120. Screening of immune human antibody phage libraries by using purified soluble gp140s that contain both gp120 and truncated gp41 lacking the transmembrane domain and cytoplasmic tail most often leads to the selection of antibodies against gp120 (see, e.g., Zhang et al., J. Immunol. Methods, 283: 17-25 (2003)). Given the ever-increasing number of people infected with HIV, there is a need for new strategies by which to identify and/or isolate antibodies that selectively bind to the conservative gp41 subunit. Furthermore, there is a need for anti-gp41 antibodies with broadly neutralizing activity against HIV, which can be used to treat, ameliorate, inhibit, or prevent HIV infections in individuals who have, or who are at risk for developing, such infections.

The invention provides such a method, as well as antibodies that bind to the conservative gp41. These and other advantages of the invention, as well as additional inventive features, will be apparent from the description of the invention provided herein.

### BRIEF SUMMARY OF THE INVENTION

The invention provides an isolated polypeptide comprising the amino acid sequence of m41H3 (SEQ ID NO: 1), m42H3 (SEQ ID NO: 2), m43H3 (SEQ ID NO: 3), m44H3 (SEQ ID NO: 4), m45H3 (SEQ ID NO: 5), m46H3 (SEQ ID NO: 6), m47H3 (SEQ ID NO: 7), m48H3 (SEQ ID NO: 8), m42 L3 (SEQ ID NO: 9), m43 L3 (SEQ ID NO: 10), m44 L3 (SEQ ID NO: 11), m45 L3 (SEQ ID NO: 12), m46 L3 (SEQ ID NO: 13), m47 L3 (SEQ ID NO: 14) and m48 L3 (also SEQ ID NO: 14), or a combination thereof, wherein the polypeptide binds with an epitope on the HIV-1 envelope glycoprotein. The invention also includes pharmaceutical compositions comprising the polypeptide, epitopes that bind to the polypeptide, and methods of using the polypeptide to inhibit HIV infection in a mammal and to detect HIV in a mammal.

Additionally, the invention provides an isolated nucleic acid molecule that encodes a polypeptide comprising m41H3 (SEQ ID NO: 1), m42H3 (SEQ ID NO: 2), m43H3 (SEQ ID NO: 3), m44H3 (SEQ ID NO: 4), m45H3 (SEQ ID NO: 5), m46H3 (SEQ ID NO: 6), m47H3 (SEQ ID NO: 7), m48H3 (SEQ ID NO: 8), m42 L3 (SEQ ID NO: 9), m43 L3 (SEQ ID NO: 10), m44 L3 (SEQ ID NO: 11), m45 L3 (SEQ ID NO: 12), m46 L3 (SEQ ID NO: 13), m47 L3 (SEQ ID NO: 14) and m48 L3 (also SEQ ID NO: 14), or a combination thereof, wherein the nucleic acid molecule is optionally in the form of a vector, wherein the nucleic acid molecule or vector is optionally contained within a host cell, wherein the polypeptide binds with an epitope on the HIV-1 envelope glycoprotein. The invention also provides pharmaceutical compositions comprising the nucleic acid molecule and methods of using the nucleic acid molecule to inhibit HIV infection in a mammal.

The invention also is directed to a method of isolating an antibody that specifically binds with an epitope of the gp41 subunit of HIV-1 envelope glycoprotein comprising: (a) providing a first composition comprising recombinant gp140, (b) providing a second composition comprising recombinant gp120, (c) labeling the recombinant gp140 of the first composition to yield a labeled first composition, (d) mixing the labeled first composition and second composition, such that the second composition is in molar excess of the labeled first composition, wherein the mixture of the labeled first and second compositions yields a third composition, (e) panning an antibody phage library with the third composition to yield antibodies that bind with the labeled gp140, (f) screening the antibodies for binding to gp140 and/or gp120 using phage ELISA, and (g) isolating an antibody that binds with an epitope of the gp41 subunit of HIV-1 envelope glycoprotein.

### DETAILED DESCRIPTION OF THE INVENTION

The invention provides polypeptides (e.g., antibodies) that bind with an epitope of the HIV-1 envelope glycoprotein (Env). The invention more specifically provides polypeptides (e.g., antibodies) that bind with the conservative gp41 subunit of the HIV-1 Env protein. Additionally, the invention provides epitopes that are recognized by the polypeptides (e.g., antibodies) of the present invention, which epitopes can be used, e.g., in the development of vaccine immunogens for the treatment or prevention of HIV.

The invention provides the selection of a panel of broadly cross-reactive monoclonal antibodies against the gp41 subunit of the HIV-1 Env using a strategy designated as competitive antigen panning (CAP). The CAP methodology is based on the use of mixtures of tagged recombinant soluble Env with truncated transmembrane domains and cytoplasmic tails (gp140) and an excess amount of untagged gp120, which facilitates the rapid identification of antibodies against epitopes on gp41.

The anti-gp41 antibodies isolated by CAP can be used for the therapy of HIV-1 infected individuals, as well as to detect HIV in an animal, including without limitation a human, or test sample. The test sample can be blood, serum, sewage, cloth, waste materials, surgical instruments, and the like. These antibodies can be also used for screening of peptide phage display libraries, libraries of Envs, and, in general, as tools for development of HIV vaccines.

The invention provides an isolated polypeptide (e.g., antibody) comprising the amino acid sequence of m41H3 (SEQ ID NO: 1), m42H3 (SEQ ID NO: 2), m43H3 (SEQ ID NO: 3), m44H3 (SEQ ID NO: 4), m45H3 (SEQ ID NO: 5), m46H3 (SEQ ID NO: 6), m47H3 (SEQ ID NO: 7), m48H3 (SEQ ID NO: 8), m42 L3 (SEQ ID NO: 9), m43 L3 (SEQ ID NO: 10), m44 L3 (SEQ ID NO: 1), m45 L3 (SEQ ID NO: 12), m46 L3 (SEQ ID NO: 13), m47 L3 (SEQ ID NO: 14) and m48 L3 (also SEQ ID NO: 14), or a combination thereof, wherein the polypeptide binds with an epitope on the HIV-1 envelope glycoprotein. Preferably, the polypeptide comprises (a) the amino acid sequences of SEQ ID NO: 2 and SEQ ID NO: 9; (b) the amino acid sequences of SEQ ID NO: 3 and SEQ ID NO: 10; (c) the amino acid sequences of SEQ ID NO: 4 and SEQ ID NO: 11; (d) the amino acid sequences of SEQ ID NO: 5 and SEQ ID NO: 12; (e) the amino acid sequences of SEQ ID NO: 6 and SEQ ID NO: 13; (f) the amino acid sequences of SEQ ID NO: 7 and SEQ ID NO: 14; and/or (g) the amino acid sequences of SEQ ID NO: 8 and SEQ ID NO: 14.

The invention also provides an isolated nucleic acid molecule that encodes a polypeptide comprising m41H3 (SEQ ID NO: 1), m42H3 (SEQ ID NO: 2), m43H3 (SEQ ID NO: 3), m44H3 (SEQ ID NO: 4), m45H3 (SEQ ID NO: 5), m46H3 (SEQ ID NO: 6), m47H3 (SEQ ID NO: 7), m48H3 (SEQ ID NO: 8), m42 L3 (SEQ ID NO: 9), m43 L3 (SEQ ID NO: 10), m44 L3 (SEQ ID NO: 1), m45 L3 (SEQ ID NO: 12), m46 L3 (SEQ ID NO: 13), m47 L3 (SEQ ID NO: 14) and m48 L3 (also SEQ ID NO: 14), or a combination thereof, wherein the nucleic acid molecule is optionally in the form of a vector, wherein the nucleic acid molecule or vector is optionally contained within a host cell. The nucleic acid molecule preferably encodes a polypeptide comprising (a) the amino acid sequences of SEQ ID NO: 2 and SEQ ID NO: 9; (b) the amino acid sequences of SEQ ID NO: 3 and SEQ ID NO: 10; (c) the amino acid sequences of SEQ ID NO: 4 and SEQ ID NO: 11; (d) the amino acid sequences of SEQ ID NO: 5 and SEQ ID NO: 12; (e) the amino acid sequences of SEQ ID NO: 6 and SEQ ID NO: 13; (f) the amino acid sequences of SEQ ID NO: 7 and SEQ ID NO: 14; and/or (g) the amino acid sequences of SEQ ID NO: 8 and SEQ ID NO: 14.

The polypeptide can be any suitable polypeptide. The polypeptide preferably is an antibody. Antibodies of the invention include both polyclonal and monoclonal antibodies. In addition to intact immunoglobulin molecules, also included are fragments or polymers of those immunoglobulin molecules, and human or humanized versions of immunoglobulin molecules or fragments thereof, as long as the molecules maintain the ability to bind with an epitope of the HIV-1 envelope glycoprotein (e.g., an epitope of the gp41 subunit of Env). The antibodies can be tested for their desired activity using the in vitro assays described herein, or by analogous methods, after which their in vivo therapeutic and/or prophylactic activities can be confirmed and quantified according to known clinical testing methods.

In a preferred embodiment, the polypeptide is a monoclonal antibody or fragment thereof. A monoclonal antibody refers to an antibody where the individual antibody within a population is identical. The monoclonal antibodies of the invention specifically include chimeric antibodies, in which a portion of the heavy and/or light chain is identical with, or homologous to, corresponding sequences in antibodies derived from a particular species or belonging to a particular antibody class or subclass, while the remainder of the chain(s) is identical with, or homologous to, corresponding sequences in antibodies derived from another species or belonging to another antibody class or subclass, as well as fragments of such antibodies, as long as they exhibit the desired antagonistic activity (see, e.g., U.S. Pat. No. 4,816,567 and Morrison et al., PNAS, 81: 6851-6855 (1984)).

The monoclonal antibodies can be made using any procedure known in the art. For example, monoclonal antibodies of the invention can be prepared using hybridoma methods, such as those described by Kohler et al., Nature, 256: 495-497 (1975).

The monoclonal antibodies also can be made by recombinant DNA methods, such as those described in U.S. Pat. No. 4,816,567. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 and U.S. Pat. No. 6,096,441.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in International Patent Application WO 94/29348 and U.S. Pat. No. 4,342,566. Papain digestion of antibodies typically produces two identical antigen-binding fragments, called Fab fragments, each with a single antigen binding site, and a residual Fc fragment. Pepsin treatment yields a fragment that has two antigen combining sites and is still capable of cross-linking antigen.

The invention encompasses chimeric antibodies and hybrid antibodies, with dual or multiple antigen or epitope specificities, single chain antibodies and fragments, such as, Fab′, F(ab′)₂, Fab, scFv, and the like, including hybrid fragments and IgGs. Such antibodies and fragments can be made by techniques known in the art and can be screened for specificity and activity according to the methods set forth in the Examples and in general methods for producing antibodies and screening antibodies for specificity and activity (see, e.g., Harlow and Lane. Antibodies, A Laboratory Manual. Cold Spring Harbor Publications, New York, (1988)).

The invention also encompasses human antibodies and/or humanized antibodies. Many non-human antibodies (e.g., those derived from mice, rats, or rabbits) are naturally antigenic in humans and, thus, can give rise to undesirable immune responses when administered to humans. Therefore, the use of human or humanized antibodies in the methods of the invention serves to lessen the chance that an antibody administered to a human will evoke an undesirable immune response.

The human antibodies and humanized antibodies of the invention can be prepared by any known technique. Examples of techniques for human monoclonal antibody production include those described by Boerner et al., J. Immunol., 147(1): 86-95 (1991). Human antibodies of the invention (and fragments thereof) can also be produced using phage display libraries (see, e.g., Marks et al., J. Mol. Biol., 222: 581-597 (1991)). The human antibodies of the invention can also be obtained from transgenic animals. For example, transgenic, mutant mice that are capable of producing a full repertoire of human antibodies, in response to immunization, have been described (see, e.g., Jakobovits et al., PNAS, 90: 2551-255 (1993); and Jakobovits et al., Nature, 362: 255-258 (1993)).

Methods for humanizing non-human antibodies are well known in the art. For example, humanized antibodies can be generated according to the methods disclosed in Jones et al., Nature, 321: 522-525 (1986); Riechmann et al., Nature, 332: 323-327 (1988); Verhoeyen et al., Science, 239, 1534-1536 (1988), by substituting rodent complementarity-determining regions (CDRs) or CDR sequences for the corresponding sequences of a human antibody. Methods that can be used to produce humanized antibodies are also described in U.S. Pat. No. 4,816,567, U.S. Pat. No. 5,565,332, U.S. Pat. No. 5,721,367, U.S. Pat. No. 5,837,243, U.S. Pat. No. 5,939,598, U.S. Pat. No. 6,130,364, and U.S. Pat. No. 6,180,377.

The polypeptides of the invention also encompass bivalent antibodies, as well as fusion molecules and conjugates with other molecules that can enhance the HIV inhibitory effect of the polypeptide. The generation of fusion molecules (e.g., proteins) and conjugates (e.g., through physical or chemical conjugation) is within the ordinary skill in the art and can involve the use of restriction enzyme or recombinational cloning techniques (see, e.g., U.S. Pat. No. 5,314,995).

The fusion molecule (e.g., protein) or conjugate can comprise one or more of SEQ ID NOs: 1-14 in combination with any suitable second molecule. For example, the fusion molecule or conjugate can comprise one or more of SEQ ID NOs: 1-14 in combination with a neutralizing scFv antibody fragment or an Fab fragment (e.g., that binds to an epitope of HIV). Alternatively, the fusion protein or conjugate can comprise CD4 or a toxin.

Toxins are poisonous substances that usually are produced by plants, animals, or microorganisms that, in sufficient dose, are preferably lethal. A preferred toxin for use in the fusion molecule or conjugate of the invention is Pseudomonas toxin, Diphtheria toxin tetanus toxoid, ricin, cholera toxin, Shiga-like toxin (SLT-I, SLT-II, SLT-IIV), LT toxin, C3 toxin, Shiga toxin, pertussis toxin, tetanus toxin, Pseudomonas exotoxin, alorin, saporin, modeccin, and gelanin, as well as other therapeutic agents.

The polypeptide (e.g., antibody) and the toxin can be linked in several ways. If the hybrid molecule is produced by expression of a fused gene, a peptide bond serves as the link between the toxin and the polypeptide. Alternatively, the toxin and the polypeptide can be produced separately and later coupled (e.g., by means of a non-peptide covalent bond). For example, the covalent linkage may take the form of a disulfide bond. In this case, the nucleic acid molecule encoding the polypeptide optionally can be engineered to contain an extra cysteine codon. The cysteine is preferably positioned so as to not interfere with the binding activity of the molecule. The toxin molecule preferably is derivatized with a sulfhydryl group reactive with the cysteine of the modified polypeptide. In the case of a peptide toxin, this optionally can be accomplished by inserting a cysteine codon into the nucleic acid molecule encoding the toxin. In another alternative, a sulfhydryl group, either by itself or as part of a cysteine residue, can be introduced using solid phase polypeptide techniques.

Moreover, the polypeptide of the invention can be combined with other well-known therapies and prophylactic vaccines already in use. The combination of the polypeptide of the invention and other therapeutic agents can provide a greater therapeutic effect than either agent alone, and preferable generate an additive or a synergistic effect with current treatments. For example, the polypeptide of the invention can be combined with other HIV and AIDS therapies and vaccines, such as highly active antiretroviral therapy (HAART), azidothymidine (AZT), structured treatment interruptions of HAART, cytokine immune enhancement therapy (interleukin (IL)-2, IL-12, CD40L+IL-12, IL-7, and interferons (IFNs)), other HIV-1 neutralizing antibodies, cell replacement therapy, recombinant viral vector vaccines, DNA vaccines, inactivated virus preparations, immunosuppressive agents, such as Cyclosporin A, and cyanovirin therapy (see, e.g., U.S. Pat. No. 6,015,876 and International Patent Application WO 03/072594). Such therapies can be administered in the manner already in use for the known treatment providing a therapeutic or prophylactic effect (see, e.g., Silvestri et al. Immune Intervention in AIDS. In Immunology of Infectious Disease. H. E. Kauffman, A. Sher, and R. Ahmed eds., ASM Press. Washington D.C. 2002)).

The polypeptide (e.g., antibody) is preferably a broadly neutralizing antibody against HIV that can inhibit the activity (e.g., the ability to enter a target cell) of HIV isolates from more than one genetic subtype or clade. The polypeptide preferably is broadly cross-reactive (e.g., can bind to a wide range of isolates from different clades). For example, the polypeptide preferably binds to an epitope of an HIV-1 envelope glycoprotein of clades A, B, C, D, E, EA, F, FB, G, H and/or O.

The polypeptide of the invention physically associates with its target molecule (e.g., gp41 of HIV-1 Env) to inhibit HIV entry into a cell and/or to inhibit or prevent HIV replication in a mammal. Preferably, the polypeptide does not substantially physically associate with other molecules. In other words, the polypeptide specifically binds, specifically reacts with, or specifically interacts with its target molecules.

The inventive HIV-1 binding polypeptide is capable of binding with HIV-1 when contacted with a solution or material comprising HIV-1 or HIV-1 envelope proteins.

The polypeptide (e.g., antibody) of the invention binds to an epitope on the HIV-1 Env (e.g., an epitope on the gp41 subunit of the HIV-1 Env). The invention, therefore, encompasses epitopes that are recognized by the polypeptides of the invention (e.g., recognized by the amino acid sequences of SEQ ID NOs: 1-14).

In a preferred embodiment, the epitopes recognized by the polypeptides (e.g., antibodies) of the invention are conformational. The antibodies of the invention do not compete with most of the mouse antibodies previously developed to map epitopes on gp41 (see, Broder et al., PNAS, 91: 11699-11703 (1994); and Broder et al., Gene, 142: 167-174 (1994)), but do compete strongly with the cluster IV antibody T3, a conformation-dependent mouse antibody (see Example 8). The epitopes recognized by the polypeptides (e.g., antibodies) of the invention, however, are different from the T3 epitope based on their binding capacity to N36/C34 formed 6-HLB (see Example 8).

While not wishing to be bound by any particular theory, the binding of the polypeptides of the invention (e.g., antibodies) may cause conformational changes in the gp41 region that negatively affect T3 binding. The competition with the cluster V antibody D3 (see Example 8) indicates that the epitopes could involve N-terminal sequences. It appears that in the three-dimensional structure of native gp41 (see FIG. 8 in Earl et al., J. Virol., 71(4): 2674-2684 (1997)), the very membrane-proximal external region (MPER) (corresponding to cluster IV antibody epitopes) is at close proximity to N-terminal heptad repeat structures (part of cluster V antibody epitopes). Thus, the epitopes recognized by the polypeptides (e.g., antibodies) of the invention are likely to comprise portions of these two regions that could be close to each other in the native gp41 conformation. The epitope length can be any suitable length (e.g., 2, 3, 4, 5, 6, 7, 8, 9, 10, about 12, about 15, about 17, about 20, about 25, about 30, or more amino acids of the gp41 subunit).

The epitopes recognized by the polypeptides of the invention can be used as vaccine immunogens, as active portions of vaccine immunogens, and as targets for inhibitors of HIV. For example, the epitopes of the invention (or polypeptides comprising the epitopes) can be used as a target to isolate antibodies, other than those of the present invention, which antibodies bind to the epitopes of the invention, and which antibodies can be used in the treatment and diagnosis of HIV.

While it is possible to administer (e.g., as a vaccine) the epitope (or polypeptide comprising the epitope) in a pure or substantially pure form, it is preferable to present the epitope as a pharmaceutical composition, formulation, or preparation. Accordingly, the invention encompasses a composition (e.g., vaccine) comprising an epitope (or polypeptide comprising the epitope) recognized by the polypeptide of the invention. The composition can further comprise one or more pharmaceutically acceptable carriers (as described herein) and, optionally, other therapeutic ingredients.

The composition comprising the epitope can be used as a vaccine either prophylactically or therapeutically. When provided prophylactically, the vaccine is provided in advance of any evidence of an active HIV infection. The prophylactic administration of the vaccine attenuates or preferably prevents, HIV infection in a mammal. In a preferred embodiment, mammals, preferably humans, at high risk for HIV infection are prophylactically treated with the vaccines of the invention. When provided therapeutically, the vaccine is provided to enhance the patient's own immune response to the antigens present due to HIV infection. The vaccine, which acts as an immunogen, optionally can be a partially or substantially purified recombinant polypeptide comprising the epitope or analog thereof. The polypeptide comprising the epitope can be conjugated with one or more lipoproteins, administered in liposomal form, or with an adjuvant. Also encompassed by the invention are methods of developing vaccines using the epitopes of the invention.

The invention is also directed to methods of inhibiting HIV infection in a mammal. The method comprises administering an effective amount of the polypeptide, nucleic acid molecule that encodes the polypeptide, vector comprising the nucleic molecule, cell comprising the nucleic acid molecule and/or vector, or compositions comprising the foregoing, to the mammal, wherein the HIV infection is inhibited.

Inhibiting a viral infection refers to the inhibition in the onset of a viral infection, the inhibition of an increase in an existing viral infection, or a reduction in the severity of the viral infection. In this regard, one of ordinary skill in the art will appreciate that, while complete inhibition of the onset of a viral infection is desirable, any degree of inhibition of the onset of a viral infection, even for a period of time, is beneficial. Likewise, one of ordinary skill in the art will appreciate that, while elimination of viral infection is desirable, any degree of inhibition of an increase in an existing viral infection or any degree of a reduction of a viral infection is beneficial. Inhibition of a viral infection can be assayed by methods known in the art, such as by the assessment of viral load. Viral loads can be measured by methods that are known in the art, for example, using polymerase chain reaction assays to detect the presence of viral nucleic acid, or antibody assays to detect the presence of viral protein in a sample (e.g., blood) from a mammal. Alternatively, the number of CD4+ T cells in a viral-infected mammal can be measured. A treatment that inhibits an initial or further decrease in CD4+ T cells in a viral-infected mammal, or that results in an increase in the number of CD4+ T cells in a viral-infected mammal, is an efficacious treatment.

The mammal can be any mammal at risk for a viral infection or infected with a virus, such as a mouse, rat, rabbit, cat, dog, sheep, cow, horse, pig, or primate. Preferably, the mammal is a human.

The polypeptide can be administered to a mammal as a polypeptide, as a nucleic acid molecule, as a vector comprising the nucleic acid encoding the polypeptide, or as a cell (e.g., a host cell) comprising any of the above. Vectors include nucleic acid vectors, such as naked DNA and plasmids, and viral vectors, such as retroviral vectors, parvovirus-based vectors (e.g., adenoviral-based vectors and adeno-associated virus (AAV)-based vectors), lentiviral vectors (e.g., Herpes simplex (HSV)-based vectors), and hybrid or chimeric viral vectors, such as an adenoviral backbone with lentiviral components (see, e.g., Zheng et al., Nat. Biotech., 18(2): 176-80 (2000); International Patent Application WO 98/22143; International Patent Application WO 98/46778; and International Patent Application WO 00/17376) and an adenoviral backbone with AAV components (see, e.g., Fisher et al., Hum. Gene Ther., 7: 2079-2087 (1996)). Vectors and vector construction are known in the art (see, e.g., Sambrook et al., Molecular Cloning: A Laboratory Manual, 2d edition, Cold Spring Harbor Laboratory, NY (1989); and Ausubel et al., Current Protocols in Molecular Biology, Green Publishing Associates and John Wiley & Sons, New York, N.Y. (1994)).

The vector can comprise any suitable promoter and other regulatory sequences (e.g., transcription and translation initiation and termination codons, which are specific to the type of host) to control the expression of the nucleic acid sequence encoding the polypeptide. The promoter can be a native or normative promoter operably linked to the nucleic acid molecule described above. The selection of promoters, including various constitutive and regulatable promoters, is within the skill of an ordinary artisan. Examples of regulatable promoters include inducible, repressible, and tissue-specific promoters. Specific examples include viral promoters, such as adenoviral promoters and AAV promoters. Additionally, combining the nucleic acid described above with a promoter is within the skill in the art.

Cells (e.g., isolated host cells) comprising the above-described polypeptide or nucleic acid molecule encoding the polypeptide, optionally in the form of a vector, are also provided by the invention. Any suitable cell can be used. Examples include host cells, such as E. coli (e.g., E. coli Tb-1, TG-1, DH5α, XL-Blue MRF′ (Stratagene), SA2821, and Y1090), Bacillus subtilis, Salmonella typhimurium, Serratia marcescens, Pseudomonas (e.g., P. aerugenosa), N. grassa, insect cells (e.g., Sf9, Ea4), yeast (S. cerevisiae) cells, and cells derived from a mammal, including human cell lines. Specific examples of suitable eukaryotic host cells include VERO, HeLa, 3T3, Chinese hamster ovary (CHO) cells, W138 BHK, COS-7, and MDCK cells. Alternatively, cells from a mammal, such as a human, to be treated in accordance with the methods described herein can be used as host cells. Methods of introducing vectors into isolated host cells and the culture and selection of transformed host cells in vitro are known in the art and include the use of calcium chloride-mediated transformation, transduction, conjugation, triparental mating, DEAE, dextran-mediated transfection, infection, membrane fusion with liposomes, high velocity bombardment with DNA-coated microprojectiles, direct microinjection into single cells, and electroporation (see, e.g., Sambrook et al., Molecular Biology: A Laboratory Manual, Cold Spring Harbor Laboratory, NY (1989); Davis et al., Basic Methods in Molecular Biology (1986); and Neumann et al., EMBO J. 1: 841 (1982)). Desirably, the cell comprising the vector or nucleic acid molecule expresses the nucleic acid sequence, such that the nucleic acid sequence is transcribed and translated efficiently by the cell.

The nucleic acid molecules, vectors, cells, and polypeptides can be administered to a mammal alone, or in combination with a pharmaceutically acceptable carrier. By pharmaceutically acceptable is meant a material that is not biologically or otherwise undesirable (i.e., the material can be administered to a mammal, along with the nucleic acid, vector, cell, or polypeptide, without causing any undesirable biological effects or interacting in a deleterious manner with any of the other components of the pharmaceutical composition in which it is contained). The carrier is selected to minimize any degradation of the agent and to minimize any adverse side effects in the mammal, as would be well-known to one of ordinary skill in the art.

Suitable carriers and their formulations are described in Remington: The Science and Practice of Pharmacy (19th ed.) ed. A. R. Gennaro, Mack Publishing Company, Easton, Pa. (1995). Pharmaceutical carriers, include sterile water, saline, Ringer's solution, dextrose solution, and buffered solutions at physiological pH. Typically, an appropriate amount of a pharmaceutically acceptable salt is used in the formulation to render the formulation isotonic. The pH of the solution is preferably from about 5 to about 8 (e.g., about 5.5, about 6, about 6.5, about 7, about 7.5, and ranges thereof). More preferably, the pH is about 7 to about 7.5. Further carriers include sustained-release preparations, such as semipermeable matrices of solid hydrophobic polymers containing the polypeptide, which matrices are in the form of shaped articles (e.g., films, liposomes, or microparticles). It will be apparent to those persons skilled in the art that certain carriers may be more preferable depending upon, for instance, the route of administration and concentration of composition being administered.

Compositions (e.g., pharmaceutical compositions) comprising the nucleic acid molecule, vector, cell, or polypeptide can include carriers, thickeners, diluents, buffers, preservatives, surface agents and the like. The compositions can also include one or more active agents, such as antimicrobial agents, anti-inflammatory agents, anesthetics, anti-viral agents, and the like. The compositions of the invention preferably are approved for use by the U.S. FDA or the equivalent in other countries.

The active agents can be any suitable active agent, including azidothymidine (AZT), Cyclosporin A, inactivated virus, interleukin (IL)-2, IL-12, CD40 ligand and IL-12, IL-7, and an interferon. Additionally, the active agent can be another HIV antibody, such as those known in the art and those disclosed herein.

The composition (e.g., pharmaceutical composition) comprising the nucleic acid molecule, vector, cell, or polypeptide can be administered in any suitable manner depending on whether local or systemic treatment is desired, and on the area to be treated. Administration can be topically (including ophthalmically, vaginally, rectally, intranasally, transdermally, and the like), orally, by inhalation, or parenterally (including by intravenous drip or subcutaneous, intracavity, intraperitoneal, or intramuscular injection). Topical intranasal administration refers to the delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation.

If the composition is to be administered parenterally, the administration is generally by injection. Injectables can be prepared in conventional forms, either as liquid solutions or suspensions, solid forms suitable for solution of suspension in liquid prior to injection, or as emulsions. Additionally, parental administration can involve the preparation of a slow-release or sustained-release system, such that a constant dosage is maintained (see, e.g., U.S. Pat. No. 3,610,795). Preparations for parenteral administration include sterile aqueous or non-aqueous solutions, suspensions, and emulsions. Examples of non-aqueous solvents are propylene glycol, polyethylene glycol, vegetable oils, such as olive oil, and injectable organic esters, such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives also can be present such as, for example, antimicrobials, anti-oxidants, chelating agents, and inert gases and the like.

Formulations for topical administration may include ointments, lotions, creams, gels, drops, suppositories, sprays, liquids, and powders. Conventional pharmaceutical carriers; aqueous, powder, or oily bases; thickeners; and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable.

Some of the compositions can potentially be administered as a pharmaceutically acceptable acid- or base-addition salt, formed by reaction with inorganic acids, such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base, such as sodium hydroxide, ammonium hydroxide, potassium hydroxide, and organic bases, such as mono-, di-, trialkyl, and aryl amines and substituted ethanolamines.

The nucleic acid molecule, vector, or polypeptides can be administered with a pharmaceutically acceptable carrier and can be delivered to the mammal's cells in vivo and/or ex vivo by a variety of mechanisms well-known in the art (e.g., uptake of naked DNA, liposome fusion, intramuscular injection of DNA via a gene gun, endocytosis, and the like).

Additionally, probiotic therapies are envisioned by the present invention. Viable host cells containing the nucleic acid molecule or vector of the invention and expressing the polypeptide can be used directly as the delivery vehicle for the polypeptide to the desired site(s) in vivo. Preferred host cells for the delivery of the polypeptide directly to desired site(s), such as, for example, to a selected body cavity, can comprise bacteria. More specifically, such host cells can comprise suitably engineered strain(s) of lactobacilli, enterococci, or other common bacteria, such as E. coli, normal strains of which are known to commonly populate body cavities. More specifically yet, such host cells can comprise one or more selected nonpathogenic strains of lactobacilli, such as those described by Andreu et al. (J. Infect. Dis., 171(5): 1237-43 (1995)), especially those having high adherence properties to epithelial cells (e.g., vaginal epithelial cells) and suitably transformed using the nucleic acid molecule or vector of the invention.

If ex vivo methods are employed, cells or tissues can be removed and maintained outside the body according to standard protocols known in the art. The compositions can be introduced into the cells via any gene transfer mechanism, such as calcium phosphate mediated gene delivery, electroporation, microinjection, or proteoliposomes. The transduced cells then can be infused (e.g., with a pharmaceutically acceptable carrier) or homotopically transplanted back into the mammal per standard methods for the cell or tissue type. Standard methods are known for transplantation or infusion of various cells into a mammal.

The exact amount of the compositions required to treat an HIV infection will vary from mammal to mammal, depending on the species, age, gender, weight, and general condition of the mammal, the nature of the virus, the existence and extent of viral infection, the particular polypeptide, nucleic acid, vector, or cell used, the route of administration, and whether other drugs are included in the regimen. Thus, it is not possible to specify an exact amount for every composition. However, an appropriate amount can be determined by one of ordinary skill in the art using only routine experimentation given the teachings herein. The dosage ranges for the administration of the compositions are those large enough to produce the desired effect; however, the dosage should not be so large as to cause adverse side effects, such as unwanted cross-reactions, anaphylactic reactions, and the like. Dosage can vary, and can be administered in one or more (e.g., two or more, three or more, four or more, or five or more) doses daily, for one or more days. The composition can be administered before viral infection or immediately upon determination of viral infection and continuously administered until the virus is undetectable.

By “effective amount” is meant the amount of a polypeptide of the invention that is useful for treating, partially or completely inhibiting, or preventing an HIV infection in a patient or subject or partially or completely inhibiting entry of HIV into a cell, as described herein. Effective dosages and schedules for administering the polypeptides of the invention may be determined empirically, and making such determinations is routine to one of ordinary skill in the art. The skilled artisan will understand that the dosage of the polypeptides will vary, depending upon, for example, the species of the subject the route of administration, the particular polypeptide to be used, other drugs being administered, and the age, condition, sex and extent of the disease in the subject as described above. An effective dose of the polypeptide of the invention generally will range between about 1 μg/kg of body weight and 100 mg/kg of body weight. Examples of such dosage ranges are (but are not limited to), e.g., about 1 μg-100 μg/kg, about 100 μg-1 mg/kg, about 1 mg/kg-10 mg/kg, or about 10 mg-100 mg/kg, once a week, bi-weekly, daily, or two to four times daily. Guidance in selecting appropriate doses for anti-HIV antibodies, such as the polypeptides of the invention, is found in the literature on therapeutic uses of antibodies (see, e.g., Handbook of Monoclonal Antibodies, Ferrone et al., eds., Noges Publications, Park Ridge, N.J., (1985); and Smith et al., Antibodies in Human Diagnosis and Therapy, Haber et al., eds., Raven Press, New York (1977)). A typical daily dosage of the polypeptide used might range from about 1 μg/kg to up to about 100 mg/kg of body weight or more per day, depending on the factors mentioned above. For example, the range can be from about 100 mg to about 1 g per dose. Nucleic acids, vectors, and host cells should be administered so as to result in comparable levels of production of polypeptides.

The invention also includes kits comprising the polypeptides, nucleic acid molecules, vectors, cells, epitopes, or compositions of the foregoing. The kit can include a separate container containing a suitable carrier, diluent, or excipient. The kit also can include an adjuvant, cytokine, antiviral agent, immunoassay reagents, PCR reagents, radiolabels, and the like. Additionally, the kit can include instructions for mixing or combining ingredients and/or administration.

The invention also provides a method of detecting HIV in a mammal comprising (a) contacting a sample obtained from the mammal with the polypeptide of the invention. If an antigen is present in the mammal (e.g., HIV-1 Env), to which the polypeptide will bind, a complex will form between the polypeptide and the antigen. Detection of the complex indicates the presence of HIV in the mammal.

The sample from the mammal can be any suitable sample to detect the presence of HIV (e.g., serum). The complex can be detected by any suitable manner. The polypeptides of the invention are utilizable as labeled molecules employed in radioimmunoassay (RIA) or enzyme immunoassay (EIA), particularly enzyme linked immunosorbent assay (ELISA), by introducing thereto radioactive substances such as I¹²⁵, I¹³¹, H³ (tritium), C¹⁴, and the like; various enzyme reagents such as peroxidase (POX), chymotripsinogen, procarboxypeptidase, glyceraldehyde-3-phosphate dehydrogenase, amylase, phosphorylase, D-Nase, P-Nase, β-galactosidase, glucose-6-phosphate dehydrogenase, ornithine decarboxylase, and the like. The radioactive substance can be introduced in a conventional manner. For example, the introduction of radioactive iodine, I¹²⁵, can be carried out by the oxidative ionization method using chloramine T (see, e.g., Hunter et al., Nature, 194: 495-496 (1962)) or by using the Bolten-Hunter reagent (I¹²⁵-iodinated p-hydroxyphenyl propionic acid N-hydroxysuccinimide ester).

The invention also provides methods, including competitive antigen panning (CAP), to isolate antibodies against gp41. Specifically, the CAP methods employ the use of soluble Envs with truncated transmembrane domains and cytoplasmic tails (gp140s). Engineered gp140s with exposed conserved region of gp41 (e.g., tethered Envs) can be used as antigens to isolate antibodies that specifically bind with an epitope to gp41. Specifically, the CAP method comprises tagging recombinant gp140 and mixing the recombinant gp140 with an excess of nontagged recombinant gp120. This mixture is used for panning of an antibody phage library, wherein the gp140 bound with (presumably) anti-gp41 antibodies is extracted.

Accordingly, the invention provides a method of isolating an antibody that specifically binds with an epitope of the gp41 subunit of HIV-1 envelope glycoprotein comprising: (a) providing a first composition comprising recombinant gp140, (b) providing a second composition comprising recombinant gp120, (c) labeling the recombinant gp140 of the first composition to yield a labeled first composition, (d) mixing the labeled first composition and second composition, wherein the mixture of the labeled first and second compositions yields a third composition, (e) panning an antibody phage library with the third composition to yield antibodies that bind with the labeled gp140, (f) screening the antibodies for binding to gp140 and/or gp120 using phage ELISA, and (g) isolating an antibody that binds with an epitope of the gp41 subunit of HIV-1 envelope glycoprotein, as described in more detail in the Examples.

The recombinant gp140 and gp120 for use in the method can be any suitable gp120 and gp140, such as gp120 and gp140 isolated from CM243, 89.6 and/or R2, (see, e.g., Chow et al., Biochem., 41: 7176-7182 (2002); Zhang et al., J. Virol., 78(17): 9233-42 (2004); and Brenneman et al., Brain Res. 838(1-2): 27-36 (1999)).

The label for use in the method can be any suitable label known in the art, such as biotinylated proteins or peptides.

In the method of isolated an antibody using CAP, the second composition preferably is in molar excess of the labeled first composition. The molar excess of the second composition is at least about 0.5, about 1, about 2, about 3, about 4, about 5, about 6, about 7, about 8, about 9, about 10, or more than the labeled composition. Preferably, the molar excess of the second composition is about 5 times that of the labeled first composition.

The following examples further illustrate the invention but, of course, should not be construed as in any way limiting its scope.

### EXAMPLE 1

This example demonstrates the isolation of anti-gp41 antibodies using competitive antigen panning (CAP).

The use of recombinant soluble Envs with truncated transmembrane domains and cytoplasmic tails (gp140s) as antigens for panning of phage libraries has yielded anti-gp120 antibodies (see, e.g., Zhang et al. J. Immunol. Methods, 283: 17-25 (2003); and Zhang et al., J. Mol. Biol. 335: 209-219 (2004)). One of the strategies to select anti-gp41 antibodies by using gp140 as antigen would be to preincubate the library with gp120, thus eliminating most of the anti-gp120 antibodies. However, such a strategy could also result in non-specific retention of many other antibodies, including anti-gp41 antibodies that would be lost for the subsequent rounds of panning.

A better strategy would be to simultaneously incubate gp140 with an excess of gp120, and then selectively extract the gp140 bound to (presumably) anti-gp41 antibodies. Under these equilibrium conditions, most of the high-affinity anti-gp120 antibodies that bind to gp120 and the anti-gp41 antibodies that bind non-specifically (with low-affinity) to gp120 will be selected against.

To test this competitive antigen panning (CAP) strategy, recombinant gp140 was biotinylated from three different isolates (CM243, 89.6, and R2 denoted as gp140_{CM243}, gp140_{89.6}, and gp140_{R2}, respectively) (as described previously in Zhang et al., J. Immunol. Methods, supra). The recombinant gp140 was mixed with 5-fold molar excess of non-biotinylated recombinant gp120.

This mixture was used for panning of an antibody phage library that was constructed using pCom3H phagemid vector and 30 cc of bone marrow obtained from three long term nonprogressors whose sera exhibited the broadest and most potent HIV-1 neutralization among 37 HIV-infected individuals (provided by T. Evans, University of California, Davis). Specifically, phage (5×10¹² cfu/ml) were preadsorbed on streptavidin-M280-Dynabeads in phosphate buffered saline (PBS) for one hour at room temperature. The phage library was incubated with 50 nM biotinylated HIV-1 envelope glycoprotein gp140_{CM243} and 250 nM non-biotinylated gp120_{CM243} (5-fold more on molar level than biotinylated gp140_{CM243}) for two hours at room temperature with gentle agitation. The panning against tethered envs gp140_{89.6} and gp140_{R2} was done in parallel the same way as panning against gp140_{CM243}. The phage library was incubated with 50 nM biotinylated gp140_{89.6} or gp140_{R2} and their non-biotinylated gp120 counterparts (5-fold more on molar level).

In a control panning, the phage library was depleted with 250 nM biotinylated gp120_{CM243} prior to incubation with 50 nM biotinylated gp140_{CM243}. Phage binding to biotinylated Env were separated from the phage library using streptavidin-M280-Dynabeads and a magnetic separator (Dynal). After washing 20 times with 1 ml of PBS containing 0.1% Tween-20 and another 20 times with 1 ml of PBS, bound phage were eluted from the beads using 100 mM Triethanolamine followed by neutralization with 1M, pH7.5 Tris-HCl.

For the second round of panning, 10 nM (2 nM for the third round) of biotinylated gp140_{CM243}, gp140_{89.6}, and gp140_{R2} and 5-fold excess of non-biotinylated gp120_{CM243}, gp120_{89.6}, and gp120_{R2}, respectively, were used as antigens. The control phage library was also panned second and third times as described above, but with decreased amount of biotinylated gp140_{CM243} antigens (10 nM for the second round and 2 nM for the third round) after depletion with five-fold more non-biotinylated gp120_{CM}243. After the third round of panning, 96 individual clones from each panned library were screened for binding to gp140/120_{CM243}, gp140/120_{89.6}, and gp140/120_{R2} by phage ELISA (as described in Zhang et al., J. Immunol. Methods., supra).

The ELISA of selected individual clones showed that the CAP resulted in a significant number of phage-displayed antibodies that bound gp140 but did not bind gp120 (designated as gp41 binders). Table 1 sets forth the results after the third round of panning against gp140s from different isolates by using CAP or gp120 prebinding for one of the isolates (CM243).

TABLE 1
Efficient selection of gp41-specific phage-displayed antibodies
by using CAP against gp140s from different isolates.

Number of individual clones

| Isolate - procedure | gp140 binders | gp41 binders |
|---|---|---|
| 89.6 - CAP | 76 | 70 |
| R2 - CAP | 21 | 14 |
| CM243 - CAP | 60 | 20 |
| CM243 - gp120 prebinding | 59 | 7 |

This example demonstrates that CAP can be used to selectively isolate anti-gp41 antibodies.

### EXAMPLE 2

This example assesses the efficiency of the CAP methodology.

Depletion with gp120_{CM243} was performed prior to panning against gp140_{CM243} as discussed in Example 1. In this case the number of gp41 binders (7) was much smaller (3-fold) compared to the number of clones selected by CAP (Table 1). Because of the labor-intensive nature of biopanning, control experiments with other gp140s from other isolates were not performed.

Of the clones described above in Example 1, 65 exhibited relatively high binding to the antigens used for their selection as measured by phage ELISA (optical density (OD) at 405 nm>1.0). Phage-displayed antibodies with high level of binding to gp140s in phage ELISA were sequenced and analyzed for similarity. DNA sequencing showed that most of these clones were identical in sequence or differed by only a few amino acid residues in the framework (see Table 2).

TABLE 2
Efficient enrichment of gp41 binders selected by CAP.

| | Number of clones with the same sequence | | | |
|---|---|---|---|---|
| | | | | gp140_{CM243}- |
| | gp140_{89.6} | gp140_{R2} | gp140_{CM243} | gp120 prebinding |
| Clone | (45) | (14) | (5) | (2) |
| m41 | 1 | 0 | 0 | 0 |
| m42 | 2 | 0 | 0 | 0 |
| m43 | 0 | 0 | 1 | 0 |
| m44 | 4 | 1 | 2 | 1 |
| m45 | 5 | 12 | 1 | 0 |
| m46 | 0 | 1 | 0 | 0 |
| m47 | 17 | 0 | 1 | 1 |
| m48 | 16 | 0 | 0 | 0 |

Table 2 indicates the number of clones with identical third complementarity-determining regions (CDRs) of their heavy chains (H3s) and light chains (L3s). In Table 2, H3s and L3s selected by each antigen are shown in parentheses. gp120 prebinding denotes depletion of gp120 binders before panning against gp140s without using CAP. In Table 2, the numbers of clones for each antibody selected by using the three different antigens and two different procedures are shown as a measure of the enrichment efficiency.

Eight clones (designated m41 through m48) have different sequences of their H3s. The L3s were also different except for those of m47 and m48 (see Table 3). The highest number of clones (45 vs 14 and 5) were selected by using the tethered gp140 from the 89.6 isolate (see Table 2). This tethered gp140 was designed to exhibit enhanced exposure of presumably conserved gp41 structures that play a role in the entry mechanism (see, e.g., Chow et al., Biochem., 41: 7176-7182 (2002)). Because of the labor-intensive nature of biopanning and the unavailability of wild type gp140 from 89.6, control comparative experiments were not performed. Such experiments could show whether tethered gp140s used as antigens for screening of immune antibody libraries provide much more efficient enrichment of gp41 antibodies compared to gp140s from wild type virus.

Two clones (m44 and m45), which were enriched by using the tethered gp140, were also selected by the other two antigens suggesting that their epitopes are shared among the three isolates. One of those (m45) was significantly enriched by using R2 suggesting enhanced exposure of its epitope on R2 gp41. Two other clones selected by the tethered gp140 (m47 and m48), were extensively enriched, but not selected by the two other antigens, except for one clone of m47, which was selected by the gp140 from the CM243 isolate.

These results suggest that CAP is an efficient methodology for selection of gp41-specific antibodies by use of the entire oligomeric Env ectodomain (gp140) and that the tethered gp140 appears to be an efficient antigen for selection of anti-gp41 antibodies from phage libraries.

### EXAMPLE 3

This example confirms the specificity of interactions of the soluble anti-gp41 antibodies with gp41 in the context of gp140.

Soluble Fab was produced from the antibodies isolated in Example 1 as described previously (Barbas et al., Phage Display: A Laboratory Manual, Cold Spring Harbor Laboratory Press, Cold Spring Harbor (2001)). Competition ELISA was performed, wherein free gp120 competed with immobilized gp140 for binding to soluble anti-gp41 antibody Fabs (namely, m43, m44, m45, m47, and m48). The anti-gp120 antibody Fab m14 (see, e.g., Zhang et al., J. Virol., 17(78), 9233-9242 (2004)) and the anti-gp41 antibodies IgG 2F5 (see, e.g., Muster et al., J. Virol., 67: 6642-6647 (1993)), IgG 4E10 (see, e.g., Stiegler et al., AIDS Res. Hum. Retroviruses, 17: 1757-1765 (2001)) and Fab Z13 (see, e.g., Zwick et al., J. Virol., 75: 10892-10905 (2001)) were used as controls.

The soluble gp120 did not compete with the anti-gp41 antibodies, thus confirming their specificity for gp41, except m48, which exhibited a slight decrease in binding to immobilized gp140 in the presence of high concentration (1 mM) of free gp120. A similar decrease was also observed with the control antibody, Z13. It is interesting to note that at a gp120 concentration of 1 mM, the binding of the control antibody, 4E10, was increased.

The specificity of these antibodies was further confirmed by their binding to gp41-Fc fusion protein. Briefly, the ectodomain of 89.6 gp41 was expressed as a fusion to immunoglobulin Fc portion. gp41-Fc at 1 μg/ml was coated on 96-well microplates. The plates were blocked using 3% BSA in PBS. Three-fold serially diluted anti-gp41 antibodies m43, m44, m45, m47, m48, and control human antibodies 2F5, 4E10, and Z13, and mouse antibody NC-1 were added to the wells. Anti-gp120 antibody m14 and BSA were used as negative controls. Bound human antibodies were revealed by using HRP-conjugated anti-human IgG, F(ab′)2, and 2,2′-azino-bis(3-ethylbenzthiazoline-6-sulfonic acid) (ABTS) as substrate. The same second antibody and ABTS were added to the wells with BSA control. Bound mouse antibody NC-1 was detected using Horseradish Peroxidase (HRP)-conjugated anti-mouse IgG and ABTS as substrate.

All tested antibodies bound to the gp41 fusion protein. m45 bound at the fusion protein at similar levels at antibody concentration levels from 0.005 to 10 μg/ml, whereas m43, m44, m47, and m48 achieved maximum binding at higher antibody concentrations (namely, about 0.014 μg/ml or higher for m44, about 0.123 μg/ml or higher for m43, and about 0.111 μg/ml or higher for m47 and m48).

### EXAMPLE 4

This example demonstrates the neutralizing activity of the anti-gp41 antibodies against selected primary HIV-1 isolates from different clades.

Although gp41 is immunogenic and the titer of anti-gp41 antibodies in HIV-1-infected humans is high (see, e.g., Opalka et al., J. Immunol. Methods, 287: 49-65 (2004)), there are only three known human monoclonal antibodies against gp41 (2F5 (Muster et al., supra), 4E10 (Stiegler et al., supra), and Z13 (Zwick et al., supra), which exhibit broad neutralizing activity. To evaluate the possibility for broad neutralizing activity of selected soluble anti-gp41 Fabs (namely, m42, m43, m44, m45, and m47), the inhibitory activity for infection of peripheral blood mononuclear cells (PBMCs) by three HIV-1 primary isolates from clades A (RW009), B (Bal), and C (BR025) was tested (see Table 3). For comparison, the potent broadly neutralizing anti-gp120 Fabs m14 (Zhang et al., J. Virol., supra) and X5 (Moulard et al., supra) were used.

The neutralization assay is based on infection of the PBMCs with infectious viruses and measurement of reverse transcriptase (RT) seven days after infection. The procedure was as follows: 100 μl of antibodies diluted in complete RPMI (Sigma-Aldrich) with Interleukin-2 (IL-2) were incubated with 50 μl of virus containing 100 TCID₅₀ for 30 minutes at 37° C. and added to 50 μl of PHA-activated PBMC (1×10⁶) in complete RPMI 1640 with IL-2. Triplicate samples were taken on day 7 for the RT assay.

TABLE 3
Neutralizing activity of anti-gp41 hmAbs against three primary
HIV-1 isolates from different clades in a PBMC assay.

| | HIV-1 Isolates | | | |
|---|---|---|---|---|
| | RW009 | Bal | BR025 | Median |
| Antibody | (Clade A) | (Clade B) | (Clade C) | values |
| Fab m42 | 52 | 70 | 72 | ND |
| Fab m43 | 63 ± 4 | 39 ± 5 | 88 ± 3 | 63 |
| Fab m44 | 82 ± 4 | 17 ± 10 | 88 ± 8 | 82 |
| Fab m45 | 92 ± 3 | 0 ± 18 | 95 ± 1 | 92 |
| Fab m47^{a} | 40 ± 1 | 0 ± 5 | 50 ± 27 | 40 |
| Fab m14^{b} | 83 ± 13 | 21 ± 3 | 92 ± 2 | 83 |
| Fab X5 | 99 ± 0 | 95 ± 3 | 97 ± 0 | 97 |

^{a}tested at 64 μg/ml
^{b}tested at 90 μg/ml
ND—not determined

In Table 3, the percentage inhibition of RT activity (mean ±standard deviation (SD)) at day 7 of a spreading HIV-1 infection of PBMCs is presented as a measure of the antibody inhibitory activity. The antibody Fab concentration was 100 μg/ml unless indicated otherwise. All five anti-gp41 Fabs neutralized BR025 (clade C) and RW009 (clade A) to various degrees comparable to the neutralizing activity of m14. Bal (clade B) was also neutralized by m42 and m43, but weakly by m44 and not neutralized by m45 and m47. Fab m14 also weakly neutralized Bal. Fab X5 neutralized all of the selected isolates with high potency as previously reported (Moulard et al., PNAS, 99: 6913-6918 (2002)).

This example demonstrates the broad neutralizing activity of the anti-gp41 antibodies of the invention.

### EXAMPLE 5

This example further demonstrates the neutralizing activity of anti-gp41 antibodies against selected primary HIV-1 isolates from different clades.

To potentially increase the Fab potency, and confer biological effector functions and long half-life in vivo, as well as to better mimic in vivo neutralization, the Fabs of m43, m44, and m48 were converted to full antibodies in an IgG1 format, and a PBMC-based assay measuring RT was used to evaluate their inhibitory activity against a range of primary isolates from different clades as described above.

Specifically, primary isolates from the following clades were used: A (92UG029), B (HT594 and SHIV 89.6p), C (92BR025, 97ZA003, 931N101, and 93MV959), D (92UG001), E (93TH073), F (93BR029), G (G3), and O (BCF03). IgG 4E10 and Fab Z13 were used as controls and Fab m48 was also tested. The percentage of RT activity (mean ±SD) in the culture supernatent of HIV-1 infected PBMCs at day 7 is presented as a measure of the antibody inhibitory activity in Table 4. The medians were calculated by using triplicate values and not directly from the means for each isolate. The antibody concentration was 50 μg/ml.

TABLE 4
Inhibitory activity of anti-gp41 antibodies against of panel of HIV-1 primary
isolates from different clades.

| | | Percentage of RT Activity (mean ± SD) | | | | | |
|---|---|---|---|---|---|---|---|
| HIV-1 isolate | Clade | IgG m43 | IgG m44 | IgG m48 | Fab m48 | Fab Z13 | IgG 4E10 |
| 92UG029 | A | 62 ± 14 | 0 ± 6 | 20 ± 14 | 43 ± 11 | 1 ± 17 | 0 ± 14 |
| 92HT594 | B | 97.3 ± 0.1 | 21 ± 17 | 65 ± 21 | 99.7 ± 0.1 | 55 ± 23 | 31 ± 9 |
| SHIV 89.6p | B | 95.6 ± 0.4 | 95.7 ± 0.3 | 83 ± 12 | 97 ± 1 | 95 ± 1 | 25 ± 13 |
| 92BR025 | C | 66 ± 4 | 72 ± 2 | 68 ± 2 | 43 ± 16 | 63 ± 3 | 58 ± 3 |
| 97ZA003 | C | 91 ± 2 | 93 ± 2 | 94 ± 1 | 72 ± 8 | 75 ± 8 | 49 ± 12 |
| 93IN101 | C | 99.0 ± 0.0 | 99.0 ± 0.1 | 99.1 ± 0.1 | 99.7 ± 0.1 | 99.0 ± 0.1 | 98.7 ± 0.1 |
| 93MW959 | C | 94 ± 1 | 97 ± 1 | 97 ± 1 | 99.8 ± 0.0 | 92 ± 2 | 53 ± 10 |
| 92UG001 | D | 55 ± 13 | 35 ± 6 | 38 ± 6 | 98.6 ± 0.2 | 49 ± 10 | 29 ± 1 |
| 93TH073 | E | 95 ± 1 | 96.6 ± 0.3 | 96.2 ± 0.1 | 99.2 ± 0.0 | 93 ± 1 | 68 ± 3 |
| 93BR029 | F | 94.1 ± 0.4 | 93 ± 2 | 91 ± 16 | 94 ± 3 | 83 ± 11 | 62 ± 12 |
| G3 | G | 94 ± 1 | 93 ± 3 | 93 ± 1 | 92.1 ± 0.4 | 85 ± 4 | 57 ± 16 |
| BCF03 | group O | 32 ± 10 | 52 ± 20 | 44 ± 16 | 99 ± 1 | 42 ± 21 | 34 ± 8 |

| | | | | | | |
|---|---|---|---|---|---|---|
| Median values | 94 | 92 | 91 | 98 | 85 | 47 |

Interestingly, all antibodies potently neutralized most of the primary isolates, except the clade A isolate 92UG029 and the clade D isolate 92UG001, which were neutralized by only two of the antibodies tested, IgG m43 and Fab m48, respectively.

The comparison between the Fab and IgG1 formats of the same antibody, m48, showed that on average the Fab appears slightly more potent than the IgG1. Specifically, a large difference in neutralizing potency was observed for isolates 92UG001 (D) and BCF03 (O). Fab m48 completely neutralized these two isolates while IgG m48 neutralized less than 50%. But for some isolates, e.g., the clade C isolates 92BR025 and 97ZA003, the IgG1 was more potent than the Fab. In this assay the Fab m48 potency was on average comparable or higher than that of Fab Z13, and the IgG m48 potency was on average higher than the IgG 4E10 potency for this panel of primary isolates.

The results suggest that anti-gp41 antibodies m43, m44, and m48 can neutralize various isolates from different clades.

### EXAMPLE 6

This example additionally demonstrates the neutralizing activity of anti-gp41 antibodies against primary HIV-1 isolates from different clades.

To evaluate the potency of the identified anti-gp41 antibodies (namely Fab m44, Fab m48, IgG m43, IgG m47, and IgG m48), the antibodies were tested with another panel of primary isolates from different clades in a PBMC-based assay by measuring p24 antigen. Briefly, the PBMCs from healthy donors were collected and resuspended at 5×10⁶ in 10 ml of RPMI 1640 medium containing 10% FBS, 5 μg of phytohemagglutinin (PHA)/ml, and 100 U of IL-2/ml, followed by incubation at 37° C. for 3 days. The PHA-stimulated cells were infected with primary HIV-1 isolates of different clades at a multiplicity of infection (MOI) of 0.01 in the absence or presence of an antibody at graded concentrations. Culture media were changed every three days. The supernatants were collected seven days after infection and tested for p24 antigen by ELISA. The percentage inhibition of p24 production and the effective concentration for 50% (IC₅₀) and 90% (IC₉₀) inhibition was calculated by using Cacusyn computer software. Fab Z13 was used as a control. The results of the assay are shown in Tables 5 and 6.

TABLE 5
Inhibitory activity (IC₅₀) of select monoclonal antibodies
on infection of PBMCs by primary HIV-1 isolates.

| HIV-1 | Genotypes | | IC₅₀ (μg/ml) (Mean ± SD) | | | | | |
|---|---|---|---|---|---|---|---|---|
| isolate | (Clade) | Biotypes | Fab m44 | Fab m48 | Fab Z13 | IgG m43 | IgG m47 | IgG m48 |
| 92RW008 | A | R5 | >40 | >40 | >40 | >40 | >40 | >40 |
| 93UG103 | A | X4R5 | >40 | >40 | >40 | >40 | >40 | >40 |
| 92US657 | B | R5 | 19 ± 5 | 19.1 ± 0.3 | 20 ± 2 | 23 ± 2 | 24.0 ± 0.1 | 17 ± 3 |
| 93IN101 | C | R5 | 24 ± 3 | 9.0 ± 0.4 | >40 | 2.4 ± 0.8 | >40 | 1.7 ± 0.4 |
| 92UG001 | D | R5 | 24 ± 8 | 14.3 ± 0.2 | >40 | 31.4 ± 0.4 | >40 | >40 |
| 93THA051 | E | X4R5 | 6.1 ± 0.9 | >40 | >40 | 15.6 ± 0.7 | >40 | >40 |
| 93THA009 | E | R5 | 1.7 ± 0.1 | >40 | >40 | 2.6 ± 0.5 | >40 | >40 |
| 93BR020 | F | X4R5 | 7 ± 2 | 22 ± 5 | >40 | 11.1 ± 0.0 | >40 | >40 |
| RU507 | G | R5 | >40 | >40 | >40 | >40 | >40 | >40 |
| BCF02 | group O | R5 | >40 | >40 | >40 | >40 | >40 | >40 |

TABLE 6
Inhibitory activity (IC₉₀) of select monoclonal antibodies
on infection of PBMCs by primary HIV-1 isolates.

| HIV-1 | Genotypes | | IC₉₀ (μg/ml) (Mean ± SD) | | | | | |
|---|---|---|---|---|---|---|---|---|
| isolate | (Clade) | Biotypes | Fab m44 | Fab m48 | Fab Z13 | IgG m43 | IgG m47 | IgG m48 |
| 92RW008 | A | R5 | >40 | >40 | >40 | >40 | >40 | >40 |
| 93UG103 | A | X4R5 | >40 | >40 | >40 | >40 | >40 | >40 |
| 92US657 | B | R5 | 28 ± 5 | 32.3 ± 0.5 | >40 | 32 ± 2 | 33.4 ± 0.0 | 31.2 ± 0.6 |
| 93IN101 | C | R5 | >40 | >40 | >40 | 9.9 ± 0.1 | >40 | 9 ± 2 |
| 92UG001 | D | R5 | 30 ± 7 | 27 ± 2 | >40 | 36.3 ± 0.2 | >40 | >40 |
| 93THA051 | E | X4R5 | 14 ± 1 | >40 | >40 | 26.3 ± 0.8 | >40 | >40 |
| 93THA009 | E | R5 | 7.2 ± 0.1 | >40 | >40 | 8.7 ± 0.1 | >40 | >40 |
| 93BR020 | F | X4R5 | 18 ± 5 | >40 | >40 | >40 | >40 | >40 |
| RU507 | G | R5 | >40 | >40 | >40 | >40 | >40 | >40 |
| BCF02 | group O | R5 | >40 | >40 | >40 | >40 | >40 | >40 |

A maximum concentration of 40 μg/ml was used. In this assay, IgG1 m43 and Fab m44 exhibited the highest neutralizing potency with IC₅₀ and IC₉₀ values lower than 40 μg/ml for 6 and 5 of the 10 tested isolates, respectively. m43 and m44 neutralized isolates from clades B, C, D, E and F. Fab m48 neutralized isolates from clades B, C, D and F. m47 and Z13 neutralized only the clade B isolate. Notably, IC₅₀ and IC₉₀ did not vary significantly.

The same clade D isolate utilized in the PBMC-based assay of Example 5, 92UG001, was included in this panel of primary isolates and the results were the same. IgG m43 and Fab m48 neutralized this isolate while IgG m48 did not. Fab m44 also neutralized this isolate while IgG m44 neutralized 40% in the PBMC-based assay of Example 5, indicating Fabs may be more potent than IgGs for this clade D isolate. Clade A isolates were not neutralized by these antibodies. The isolates from clade G and group 0 were also not neutralized by these antibodies.

These results suggest that the newly identified anti-gp41 antibodies exhibit neutralizing activity against a panel of primary isolates from different clades with a potency on average comparable to or higher than that of Fab Z13 and IgG14E10, and that the potency of the antibodies in IgG1 format is not significantly different or is slightly lower than the potency of the Fabs.

### EXAMPLE 7

This example similarly demonstrates the neutralizing activity of anti-gp41 antibodies against selected primary HIV-1 isolates from different clades.

A pseudovirus assay was used to evaluate the inhibitory activity against a range of primary isolates. Fab Z13, scFv m6, and scFv m9 (Zhang et al., J. Mol. Biol., supra) were used for comparison.

The pseudotype virus neutralization assay was performed in triplicate by using a luciferase reporter HIV-1 Env pseudotyping system and HOS CD4+ CCR5+ or HOS CD4+ CXCR4+ cells as previously described (Zhang et al., J. Immunol. Methods, supra). The degree of virus neutralization by antibody was achieved by measuring luciferase activity as described previously (Zhang et al., J. Mol. Biol., supra).

The results of the pseudovirus-based assay are presented in Table 7. The percentage inhibition of luciferase activity is presented as a measure of the antibody inhibitory activity. The antibody Fab concentration was 100 μg/ml unless indicated otherwise.

TABLE 7
Neutralizing activity of anti-gp41 hmAbs against primary HIV-1
isolates from different clades in a pseudovirus-based assay.

| HIV-1 | | % Neutralization of anti-gp-41 Fabs at 100 μg/ml | | | | | | | | |
|---|---|---|---|---|---|---|---|---|---|---|
| Isolate | Clade | m43 | m44 | m45 | m46 | m47 | m48 | Z13 | m6* | m9* |
| 92UG037.8 | A | 0 | 0 | 42 | 8 | 8 | 1 | 0 | 78 | 50 |
| JR-CSF | B | 24 | 66 | 36 | 15 | 42 | 39 | 44 | 97 | 98 |
| BAL | B | 55 | 32 | 31 | 33 | 43 | 51 | 55 | 99 | 99 |
| MN-P | B | 7 | 26 | 3 | 5 | 3 | 50 | 19 | 98 | 99 |
| 89.6 | B | 17 | 0 | 19 | 0 | 41 | 56 | 31 | 90 | 94 |
| 92HT593.1 | B | 61 | 46 | 60 | 44 | 80 | 78 | 52 | 93 | 87 |
| GX-C44 | C | 62 | 71 | 54 | 31 | 68 | 49 | 29 | 89 | 88 |
| Z2Z6 | D | 35 | 36 | 33 | 10 | 47 | 37 | 16 | 99 | 99 |
| CM243 | E | 67 | 54 | 58 | 51 | 63 | 59 | 64 | 68 | 70 |

*25 μg/ml

The results suggest that overall anti-gp41 antibodies m43-m48 can neutralize various isolates from different clades, although at the relatively high concentration of 100 μg/ml. Their neutralizing activity is, on average, comparable to that of the broadly neutralizing antibody Fab Z13, but weaker than the potency of the CD41 antibody scFvs m9 and m6 (Zhang et al., J. Mol. Biol., supra).

### EXAMPLE 8

This example demonstrates the characterization of the epitopes of the identified anti-gp41 antibodies.

The epitopes of the three known broadly neutralizing antibodies, 2F5, 4E10 and Z13, are localized in the membrane-proximal external region (MPER) of gp41 and include stretches of known sequences. To determine whether the newly selected neutralizing antibodies bind to the same region and to begin to characterize their epitopes, four different approaches were utilized: (1) binding to peptides from different regions of gp41, (2) binding to native and denatured gp140s, (3) binding to N36/C34 formed six helix bundle (6-HLB) and single chain 5-helix bundle (5-HLB), and (4) competition for binding to gp140 with anti-gp41 antibodies of known epitopes.

(1) Binding to peptides from different regions of gp41. Thirty-four peptides derived from gp41, including N36, C34, DP178, 4E10/Z13 binding peptide 2031, and six peptides derived from the MPER, were used in an ELISA assay to test m43, m44, m45, m47, and m48 binding. Specifically, 4 μg/ml of peptide was coated on 96-well plates by incubation at 4° C. overnight. The plates were blocked and three-fold serially diluted anti-gp41 antibodies with starting concentrations of 20 μg/ml were added to the wells. None of these antibodies bound specifically to any significant degree to the peptides, indicating that the antibodies recognized conformational epitopes. This was confirmed by the observation that they bound to native but not to denatured gp140.

(2) Binding to native and denatured gp140s. To determine if denaturing factor (boiling or reducing) affects the binding of the anti-gp41 antibodies, two types of denatured gp140s were prepared: by boiling only and by adding a reducing reagent only (5 mM DTT). Briefly, 1 μg/ml of tethered gp140_{89.6} or denatured gp140_{89.6} (by boiling or reducing agent) was coated on 96-well plates. The plates were blocked using 3% BSA in PBS, and three-fold serially diluted anti-gp41 antibodies with starting concentration of 10 μg/ml were added to the wells. Bound antibodies were detected using HRP-conjugated anti-human IgG, F(ab′)2 and ABTS as substrate. Optical density at 405 nm was measured after color development at room temperature for 30 minutes.

Boiling of gp140 did not affect binding of m43, m44, m45, m47, and m48 to gp140, but addition of 5 mM DTT abolished the binding, indicating that disulfide bonds are important for the structural integrity of their epitopes. The binding of control antibodies 2F5 and Z13 was not affected by boiling or by the addition of the reducing reagent. These results suggest that the new anti-gp41 antibodies recognize conformational epitopes on gp41, which are different from those of the known broadly HIV-1-neutralizing anti-gp41 antibodies 2F5, 4E10, and Z13.

(3) Binding to N36/C34 formed six helix bundle (6-HLB) and single chain 5-helix bundle (5-HLB). To investigate the possibility that the antibodies bind to fusion intermediate structures, their binding to N36/C34 formed 6-HLB and single chain protein of 5-HLB were measured.

To determine the binding of anti-gp41 antibodies with N36/C34 formed 6-HLB, 2 μg/ml of tethered gp140_{89.6} was coated on 96-well plates. The plates were blocked with 3% BSA in PBS and two-fold serially diluted N36/C34 formed 6-HLB (prepared by mixing 20 μM N36 with 20 μM C34 and incubating the mixture at 37° C. for 30 minutes) was added to the wells. Anti-gp41 antibodies, m43, m44, m45, m47, and m48, and control human antibodies Z13 and 2F5, and control mouse antibodies NC-1 and T3 (see Earl et al., supra) were simultaneously added to the wells. The plates were incubated at 37° C. for 2 hours. Bound antibodies were revealed as described above.

In a competition ELISA with N36/C34-formed 6-HLB for binding to gp140, free 6-HLB did not compete off antibodies for binding to coated gp140, indicating that these antibodies do not bind to 6-HLB, a hairpin structure. Mouse antibody T3 and 6-HLB-specific mouse antibody NC-1 were used as controls and their binding to gp140 decreased in the presence of free 6-HLB, indicating their binding to 6-HLB. Unlike mouse antibodies T3 and NC-1, none of the newly identified anti-gp41 antibodies bound to N36/C34-formed 6-HLB.

To determine binding of anti-gp41 antibodies to single chain 5-helix bundle (5-HLB), single chain 5-HLB at 1 μg/ml was coated on microwell plates. The plates were blocked with 3% BSA in PBS and three-fold serially diluted antibodies were added to the wells. Bound antibodies were detected as described above.

In the binding assay to single chain 5-HLB, m44 and m45 showed binding capacity that was comparable to the binding of mouse antibodies T3 and NC-1. Other antibodies m43, m47, m48 and control antibodies 2F5 and Z13 did not bind to 5-HLB except the non-specific binding of Z13 and m43 at the highest antibody concentration tested (40 μg/ml). These data suggest that the new anti-gp41 antibodies can be divided into two groups based on their binding ability to 5-HLB.

(4) Competition for binding to gp140 with anti-gp41 antibodies of known epitopes. To localize the epitopes of the antibodies, anti-gp41 antibodies that recognize different regions of gp41 were used in a competition ELISA. Microwell plates were coated with 1 μg/ml of the antibodies (namely, Fab m43, Fab m44, Fab m45, Fab m47, and Fab m48) by incubation at 4° C. overnight. The plate was washed with PBS, blocked with 3% BSA at 37° C. for 1 hour, and incubated at 37° C. for 2 hours with three-fold serially diluted competing antibodies and biotinylated gp140_{89.6} at a concentration that leads to 70% maximum binding to the coated antibodies. Bound gp140_{89.6} was measured by HRP-avidin and ABTS substrate. The percentage inhibition of the binding in the presence of competing antibodies at 12 μg/ml, which is on average 10-fold higher than the EC₅₀ for the tested antibodies, is shown in Table 8.

TABLE 8
Competition of anti-gp41 antibodies.

| Competing/ | | | | | |
|---|---|---|---|---|---|
| coated | Inhibition, % | | | | |
| Antibodies | Fab m43 | Fab m44 | Fab m45 | Fab m47 | Fab m48 |
| Fab m43 | 92 | 90 | 74 | 73 | 78 |
| Fab m44 | 87 | 83 | 86 | 96 | 100 |
| Fab m45 | 89 | 88 | 100 | 86 | 100 |
| Fab m47 | 47 | 38 | 42 | 100 | 100 |
| Fab m48 | 51 | 85 | 38 | 71 | 98 |
| IgG 2F5 | 51 | 38 | 0 | 31 | 74 |
| IgG 4E10 | 29 | 42 | 6 | 10 | 63 |
| Fab Z13 | 72 | 57 | 0 | 41 | 87 |
| IgG T3 | 100 | 100 | 88 | 68 | 100 |
| IgG D3 | 29 | 42 | 5 | 30 | 75 |

As shown in Table 8, m43, m44, m45, m47 and m48 competed strongly with one another and with the mouse cluster IV antibody T3. m43, m44, m47, and m48 competed partially with 2F5, 4E10, Z13 and the mouse cluster V antibody D3, which recognizes an N-terminal region. Additionally, in a related experiment, it was determined that m43, m44, m45, m47 and m48 did not compete with M10 and D61 (cluster I); D17, D40 and D50 (cluster II); T30 (cluster VI), a control mouse antibody against the V3 loop (D47); and another control antibody against the gp120 CD4bs (m14).

These results indicate that the binding of these antibodies to env depends on the overall structure of gp41. m48 seems more sensitive to gp41 conformation compared to other antibodies. The epitopes of m43, m44, m47 and m48 may partially involve the MPER and the N-terminal region. m44 and m45 epitopes may also involve the N-heptad repeat exposed in context of C-heptad repeat for their binding to 5-HLB, a prehairpin intermediate structure. Generation of site-directed mutants of gp41 and cocrystallization of these antibodies with gp140 will provide further information about the epitopes of new anti-gp41 antibodies.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An isolated polypeptide comprising the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, or a combination thereof, wherein the polypeptide binds with an epitope on the HIV-1 envelope glycoprotein.

2. The isolated polypeptide of claim 1, comprising the amino acid sequences of
(a) SEQ ID NO: 2 and SEQ ID NO: 9;
(b) SEQ ID NO: 3 and SEQ ID NO: 10;
(c) SEQ ID NO: 4 and SEQ ID NO: 11;
(d) SEQ ID NO: 5 and SEQ ID NO: 12;
(e) SEQ ID NO: 6 and SEQ ID NO: 13;
(f) SEQ ID NO: 7 and SEQ ID NO: 14; or
(g) SEQ ID NO: 8 and SEQ ID NO: 14.

3.-8. (canceled)

9. The isolated polypeptide of claim 1, wherein the polypeptide is a monoclonal antibody or fragment thereof.

10. The isolated polypeptide of claim 1, wherein the polypeptide is an Fab, Fab′, F(ab′)₂, scFv, fusion molecule, or conjugate.

11. The isolated polypeptide of claim 1, wherein the isolated polypeptide binds with an epitope of the gp41 subunit of the HIV-1 envelope glycoprotein.

12. The isolated polypeptide of claim 1, wherein the isolated polypeptide binds with an epitope from more than one clade of HIV-1.

13. A pharmaceutical composition comprising the isolated polypeptide of claim 1 and a pharmaceutically acceptable carrier.

14. The composition of claim 13, wherein the composition further comprises an additional active agent.

15. The composition of claim 14, wherein the additional active agent is selected from the group consisting of azidothymidine (AZT), Cyclosporin A, inactivated virus, interleukin (IL)-2, IL-12, CD40 ligand and IL-12, IL-7, and an interferon.

16. An isolated polypeptide that comprises an epitope that binds with the isolated polypeptide of claim 1.

17. The isolated polypeptide of claim 16, wherein the epitope is an epitope of the gp41 subunit on the HIV-1 envelope glycoprotein.

18. A composition comprising the isolated polypeptide of claim 16 and a pharmaceutically acceptable carrier.

19. The composition of claim 18, wherein the composition is a vaccine for HIV-1.

20. An isolated nucleic acid molecule that encodes a polypeptide of claim 1.

21.-31. (canceled)

32. A vector comprising the isolated nucleic acid molecule of claim 20.

33. A cell comprising the vector of claim 32.

34. A pharmaceutical composition comprising the isolated nucleic acid of claim 20 and a pharmaceutically acceptable carrier.

35. The composition of claim 34, wherein the composition further comprises an additional active agent.

36. (canceled)

37. A method of inhibiting an HIV infection in a mammal, which method comprises administering to a mammal in need thereof an effective amount of the polypeptide of claim 1, wherein the HIV infection is inhibited.

38. A method of inhibiting an HIV infection in a mammal, which method comprises administering to a mammal in need thereof an effective amount of the pharmaceutical composition of claim 13, wherein the HIV infection is inhibited.

39. A method of inhibiting an HIV infection in a mammal, which method comprises administering to a mammal in need thereof an effective amount of the isolated nucleic acid molecule of claim 20, optionally in the form of a vector, wherein the nucleic acid molecule or vector is optionally contained within a host cell, wherein the HIV infection is inhibited.

40. (canceled)

41. The method of claim 37, wherein the mammal is a human.

42. The method of claim 37, wherein the polypeptide binds with an epitope of more than one clade of HIV.

43. A method of detecting HIV in a mammal comprising (a) contacting a sample obtained from the mammal with the polypeptide of claim 1, thereby forming a complex of the polypeptide with an antigen of the mammal, and (b) detecting the complex, whereupon detection of the complex indicates presence of HIV in the mammal.

44. A method of isolating an antibody that specifically binds with an epitope of the gp41 subunit of HIV-1 envelope glycoprotein comprising: (a) providing a first composition comprising recombinant gp140, (b) providing a second composition comprising recombinant gp120, (c) labeling the recombinant gp140 of the first composition to yield a labeled first composition, (d) mixing the labeled first composition and second composition, wherein the mixture of the labeled first and second compositions yields a third composition, (e) panning an antibody phage library with the third composition to yield antibodies that bind with the labeled gp140, (f) screening the antibodies for binding to gp140 and/or gp120 using phage ELISA, and (g) isolating an antibody that binds with an epitope of the gp41 subunit of HIV-1 envelope glycoprotein.

45. The method of claim 44, wherein the second composition is in molar excess of the first composition in the third composition.
